(19) United States
(12) Patent Application Publication
(10) Pub. No.: US 2026/0258069 A1
(43) Pub. Date: Sep. 3, 2026

(54) INTERGLYCAN ESTERS OF SUCROSE DICARBOXYLATE AND FRUCTOFURANURONATE, PROCESS FOR THE MANUFACTURE THEREOF, AND USE THEREOF AS A HYPOGLYCEMIC SLOW SUGAR

(71) Applicant: Abdessattar BOUGOUFFA, SFAX (TN)

(72) Inventor: Abdessattar BOUGOUFFA, SFAX (TN)

(21) Appl. No.: 18/880,069

(22) PCT Filed: Jul. 14, 2022
(86) PCT No.: PCT/TN2022/050004

§ 371 Date: Dec. 30, 2024

(30) Foreign Application Priority Data
Jun. 28, 2022 (TN) TN20220176

(51) Int. Cl. C07H 13/10; A23L 27/30; A23L 33/21; C07H 1/00

(52) U.S. Cl. CPC C07H 13/10; A23L 27/33; A23L 33/21; C07H 1/00

(57) ABSTRACT

The present invention relates to a novel granulated or powdered brown natural slow sugar and a process for manufacturing the same based on esterification. The product obtained, i.e. the novel brown natural slow sugar, can be consumed by anyone regardless of their state of health because it is hypoglycemic, having a glycemia of less than 1 gram. The present invention is based on obtaining a novel brown natural slow sugar by esterification characterized by slow limited athermal reactions, said sugar containing a whole range of dried plant products with health benefits. The novel brown natural sugar obtained according to the present invention can be used in any food preparations for which commercial white natural sugar is normally used, such as cakes, pastries, confections, sorbets and ice cream, juices, all types of beverages, chocolate-based preparations, caramel-based cocoa preparations. The novel brown natural slow sugar can be consumed without having to worry about dental cavities, obesity, retinopathy and other pathologies.

The present invention relates to a new natural brown slow sugar granulated or powdered characterized by the fact that it is hypoglycemic, and a process for its manufacture. Its production is achieved through an industrial manufacturing process based on esterification using commercial natural brown sugar and including a set of dried plant products, in accordance with the figure below, which includes the new molecule of the new slow natural brown sugar.

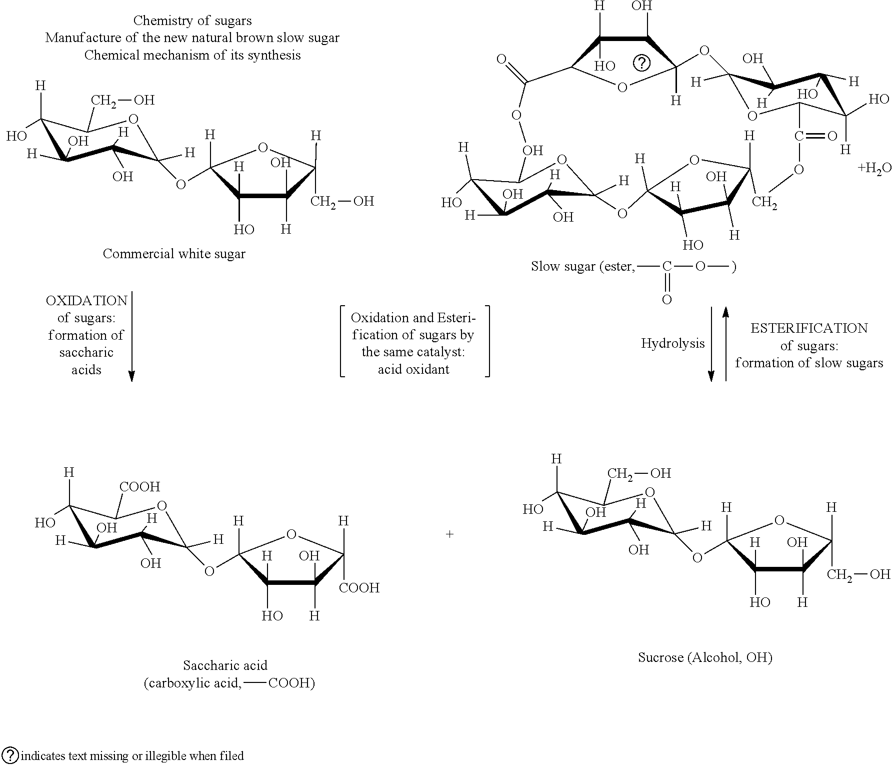

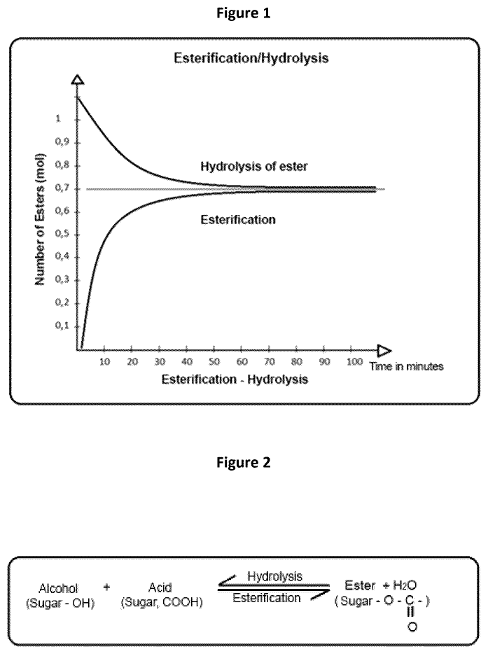

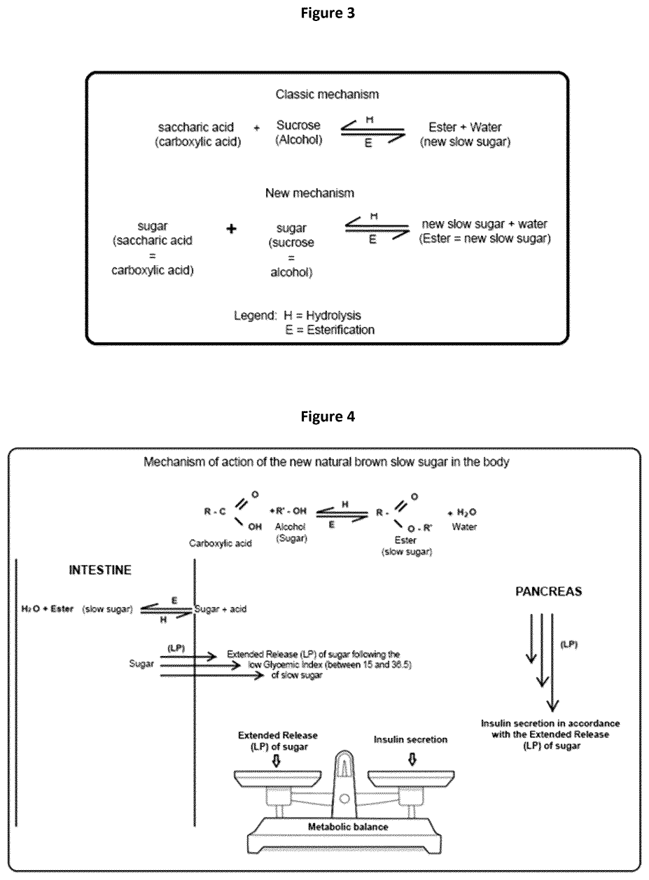

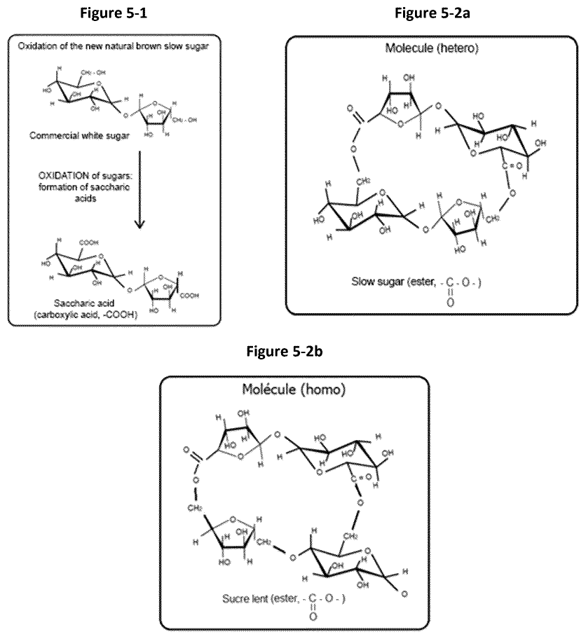

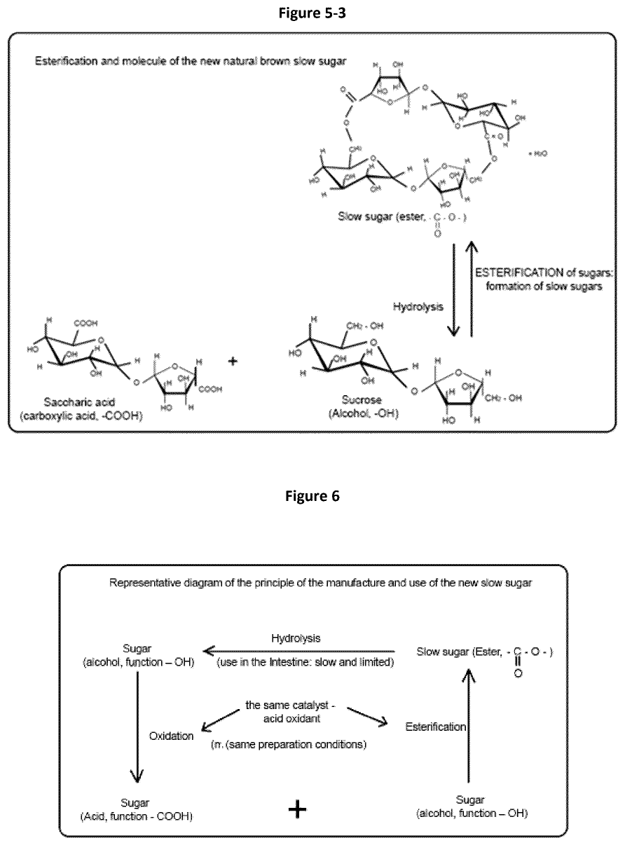

Esterification is a chemical reaction between carboxylic acid and an alcohol that leads to the formation of esters and water. (FIG. 1), (FIG. 2), (FIG. 3) and (FIG. 5).

Numerous experimental studies have shown that an equimolar mixture of a carboxylic acid and an alcohol leads to a slow and incomplete transformation.

The esterification reaction of the present invention is described as slow, limited and athermal.

The characteristic of the esterification/hydrolysis equilibrium is the dynamic equilibrium in which the two reverse reactions occur simultaneously and the reactants and products coexist in constant proportions. In fact, the two reactions evolve towards the same equilibrium state.

The present invention is based on obtaining a new natural brown slow sugar by esterification characterized by slow, limited and athermal reactions.
Slow since it is characterized by a release of new slow sugar at the level of the intestine and this in concordance with the slow secretion of insulin in the case of type 2 diabetes.
Limited by their reverse reactions since esterification is limited by the hydrolysis of the ester formed.
Athermal because the temperature does not influence the equilibrium.

The new natural brown slow sugar, granulated or powdered, of the present invention is obtained from commercial white natural sugar, by esterification on an industrial scale and this, by the addition of a main acid oxidant ingredient and the association of dried plants and seeds namely, flax, nigella, ginger, curcumin, cinnamon, clove, fenugreek, tea, coffee, cocoa, and pepper, demineralized water, following a manufacturing process, having made it possible to obtain a new natural brown slow sugar acting on consumption by hydrolysis (Fig.

Esterification on one side and hydrolysis on the other have achieved a chemical balance in the body, thus making it possible to control consumption by the limited nature of the absorption of this sugar which is controlled by the chemical balance resulting from the esterification/hydrolysis ratio.

So, if we consume more sugar than the body needs, the excess sugar is not stored but rather eliminated because esterification and hydrolysis perform the permanent control function.

This invention relates to a new natural brown slow sugar, the principle of its manufacture of which is based on the chemical balance of esterification of an alcohol and its hydrolysis.

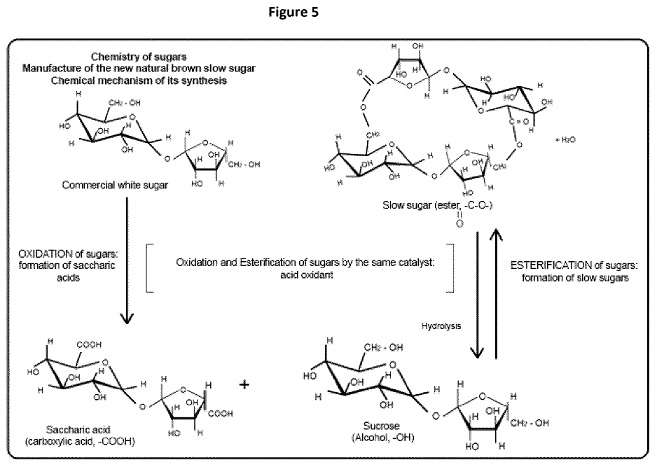

The chemical equilibrium between esterification and hydrolysis is for esterification (FIG. 1), (FIG. 2), (FIG. 3) and (FIG. 4):

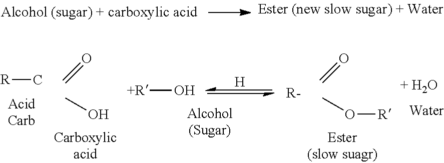

The reaction is a dynamic equilibrium between two reactions (esterification/hydrolysis) that are in opposite directions to each other and occur simultaneously.

In the industrial scale production of new natural brown slow sugar, the direction of esterification is favoured.

On the other hand, at the intestinal level, it is the opposite, since it is the hydrolysis reaction which is favored to slowly release (LP) the sugar from the new natural brown slow sugar, namely the sugar ester, and this in accordance with the sequential, slow and limited release of insulin (FIG. 4).

The sustained release LP of sugar from the new brown natural slow sugar of the present invention at the intestinal level is in agreement and similarity with the slow secretion of insulin in the case of diabetes. (FIG. 4).

The chemical basis of the new natural brown slow sugar of the present invention is obtained by the chemical mechanism of the synthesis of the new slow sugar:

### Oxidation (FIG. 5-1):

For sugars, there is firstly a reaction of monosaccharides consisting of an oxidation of saccharic acids making it more easy for the attack of primary OH groups to occur and which are generally oxidized entirely to the carboxylic acid state.

The resulting product is a polyhydroxy carboxylic acid called saccharic acid. For example, nitric acid is a good oxidizing agent for the preparation of saccharic acids.

Oxidation of the new natural brown slow sugar

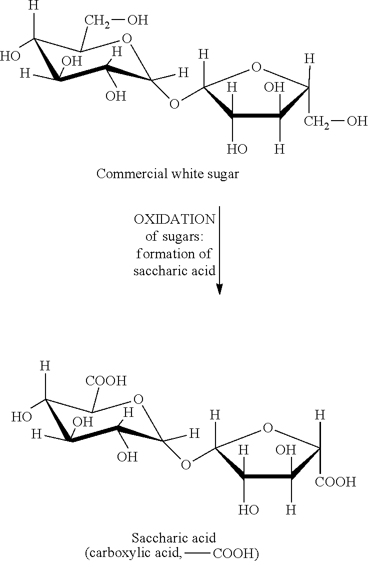

### Esterification (FIG. 5-3):

Hydroxy groups in sugars can be esterified by normal methods (Carboxylic Acid+Alcohol).

Carboxylic acids cause reactions involving the carbon of the carboxyl group, by acid-catalyzed nucleophilic addition.

The best known reaction is Esterification, which is a balanced reaction.

Starting from an equimolar mixture of carboxylic acid and alcohol, we obtain 65% ester (in number of moles) and water and 35% acid and alcohol. This yield can be improved by increasing the concentration of a reactant or by decreasing that of a product (Le Chatelier's Law).

Acid-catalyzed esterification is an important method for the preparation of esters (new slow sugar).

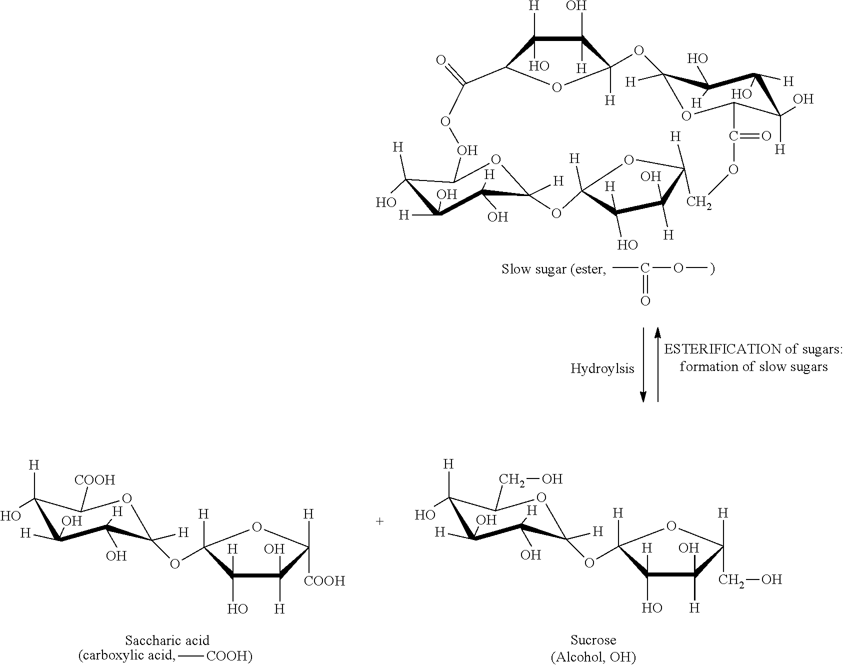

The new natural brown slow sugar of the present invention is a slow sugar, because it is obtained and consumed according to the physicochemical characteristics of the “Esterification/hydrolysis” chemical balance that are:
Slow because the release is a prolonged LP
Limited by the reverse reaction
Athermal: the temperature does not modify the equilibrium.
   the new natural brown slow sugar of the present invention is chemically in the form of esters.

Compared to natural products such as stevia, tannin and apple plants which contain esters, it is concluded that chemicals in the form of esters are not harmful.

### Extension of the Esterification Reaction (FIG. 10):

Obtaining an ester sugar following the esterification reaction between two types of sugar:
sugar alcohol (function —OH): commercial natural white sugar.
acidic sugar (function —COOH): saccharic acid.

Following the hydrolysis of sucrose into glucose and fructose, these last two sugars in turn undergo the esterification reaction.

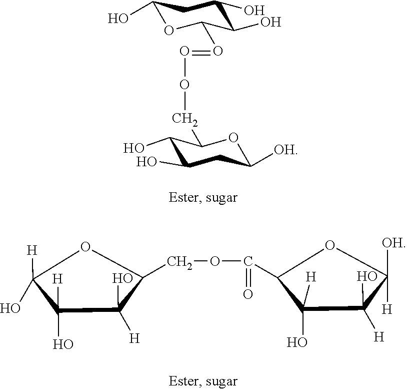

The oxidation of glucose into gluconic acid then the esterification between glucose and gluconic acid into an ester of two glucose molecules:

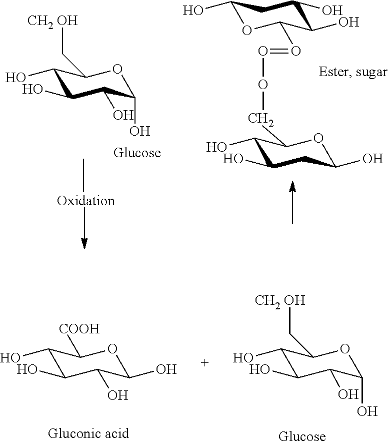

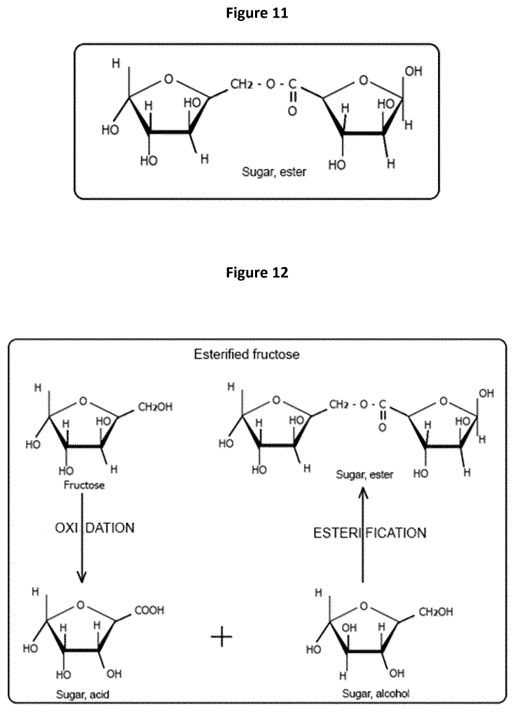

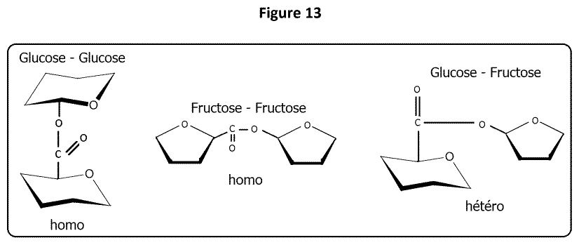

In the same way, the ester sugar for fructose is obtained (FIG. 11).

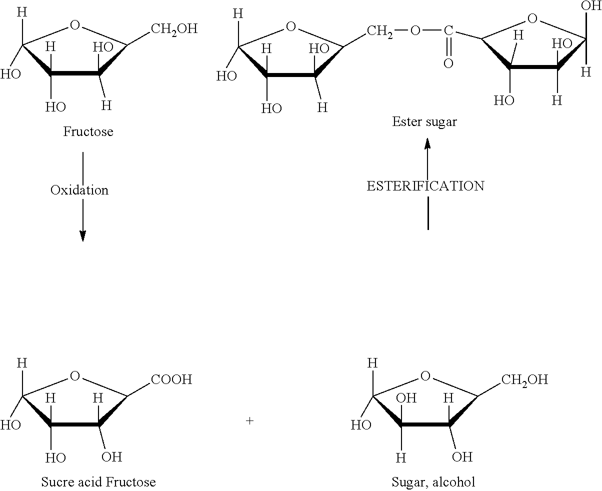

It was long wrongly believed that slow sugars were complex carbohydrates.

The new natural brown slow sugar obtained from this invention can be used in all food preparations, usually using commercial white natural sugar, such as cakes, pastries, confectionery, sorbets and ice creams, juices, drinks of all kinds, chocolate-based preparations, cocoa-based preparations, caramel-based preparations.

The new natural brown slow sugar is essential for sports and intense sports that require endurance, whatever their nature, because it is slow to be ingested into the body, ensuring the maintenance of the energy necessary for athletes and therefore it is an anti-cramping product.

In fact, the new natural brown slow sugar of this invention is assimilated slowly and therefore remains available for the duration of the effort.

The new natural brown slow sugar of this invention does not cause reactive hypoglycemia, on the other hand, commercial natural white sugar has a rapid release and causes a rapid rise in blood sugar levels, which is considered dangerous (hyperglycemia).

Slow sugars are good for your health, these slow sugars are hypoglycemic.

If we consume slow sugars, insulin will be slowly released without causing glycemic peaks.

As a result, the muscles will be supplied for several hours, which can provide good, long-lasting energy when playing sports.

The new natural brown slow sugar being prepared by chemical esterification, the hydrolysis of esters during ingestion is therefore a slow reaction which is strongly catalyzed by acids.

In this invention, in the primary and secondary alcohol esters, the mechanism is the reverse of that of acid-catalyzed esterification.

Esterification/hydrolysis are therefore in reaction equilibrium.

The production of the new natural brown slow sugar is in accordance with the process described below.

To obtain, for example, a quantity of 10 kg of new natural brown granulated slow sugar, it is necessary to make a mixture of demineralized water with an acid oxidizing catalyst and a mixture of dried plants and seeds, namely flax, nigella, ginger, curcumin, cinnamon, cloves, fenugreek, tea, coffee, cocoa, and pepper, in a tank and heat it to a temperature of 90 to 100 degrees Celsius while stirring.

Gradually add to this mixture existing in the tank which continues to remain on a fire, 10 kg of commercial white natural sugar for about 10 minutes, mixing simultaneously, with the progressive flow of sugar, using the propeller or blade stirrer, fixed and operating electrically, at a speed of 25 to 35 revolutions/minute, until a dark brown molasses is obtained. This operation, called cooking, lasts between 30 and 45 minutes.

After obtaining this molasses, the fire is then extinguished and the drying operation takes place for this molasses which is agitated by the propellers or blades of the mixer at a speed of 30 to 50 revolutions/min, under the effect of blowing fresh air followed by blowing hot air for a period of 30 to 35 min.

At the end of this drying operation, this molasses then becomes a new natural brown granulated slow sugar which will be subjected to sieving to obtain the new sugar with a uniform granulometry. Sieving results in a weight loss of 300 to 400 grams on the initial 10 kg, identifiable by the final weighing of the new natural brown slow sugar obtained from the present invention.

The nature of plants and seeds can be changed by removing or adding some.

In case of need for production of the new natural brown slow sugar in powder form, it is necessary to finish the manufacturing process described, by grinding this new granulated natural brown slow sugar obtained after sieving.

The preparation of larger quantities must imperatively observe the same process in terms of time, temperatures and speed listed above, at all stages regardless of the weight of the new natural brown slow sugar to be produced, provided that one or more tanks are available to measure the desired quantity to be produced, equipped with an adequate number of agitators capable of reaching, by agitation, the entire quantity existing in the tank(s). It is also possible to produce a large quantity by using several tanks with a capacity of 10 or 20 kg or more, operated by a control triggering simultaneous production.

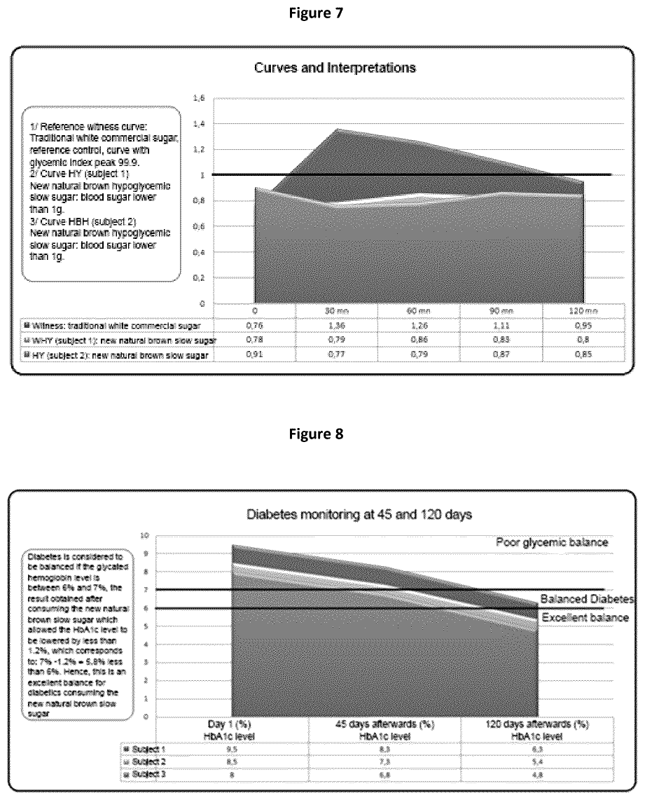

The new natural brown slow sugar of the present invention has a hypoglycemic character represented by curves reflecting the analyses of diabetic monitoring at 45 and 120 days (FIG. 7) and (FIG. 8).

The new natural brown granulated or powdered slow sugar of this invention is dietetic, calorie-free, anti-obesity and prevents dental caries.

It is also antidiabetic and therefore prevents retinopathy, and nephropathy, among others, because the new natural brown slow sugar of the present invention is esterified and it is characterized by a prolonged release LP under the effect of hydrolysis, the reverse chemical reaction of esterification.

Its consumption is therefore intended for healthy subjects and also for diabetics.

Thus a blood HbA1C level of 7% corresponds to an average blood sugar level of 1.5 g per litre of blood and 1% more or less HbA1C represents an average increase or decrease in blood sugar level of 0.30 g/litre.

For the new natural brown granulated or powdered slow sugar of this invention the following result is obtained:

$$\frac{0.24 \times 0.3}{1} = 0.072 ,$$

which clearly shows that the new natural brown slow sugar decreases HbA1c level by 7.2%

For natural white commercial sugar and for comparison purposes we obtain the following result:

$$\frac{0.32 \times 0.3}{1} = 0.092 ,$$

which that commercial white natural sugar increases HbA1c level by 9.2%

Slow sugars have long been considered to be replacement sugars and not substitute sugars or fake sugars and are often presented in liquid form, except that their preparation has not adopted another manufacturing process resembling or identical to the process of the present invention, which process is based on the chemical equilibrium “esterification/hydrolysis”.

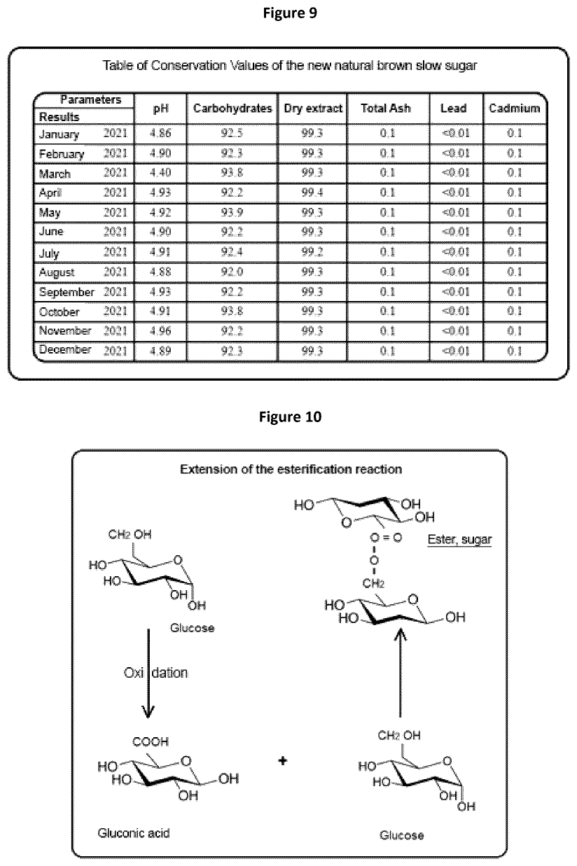

The physicochemical characteristics of the new natural brown granulated or powdered slow sugar of the present invention, obtained following analyses carried out over 12 months, are shown in the table (FIG. 9) which confirms the chemical stabilization of the slow sugar. This slow sugar is a concentrated medium that can be stored for a long time without deteriorating.

| Parameters | | Carbo- | Dry | Total | | |
|---|---|---|---|---|---|---|
| Results | pH | hydrates | extract | Ash | Lead | Cadmium |
| January 2021 | 4.86 | 92.5 | 99.3 | 0.1 | <0.01 | 0.1 |
| February 2021 | 4.90 | 92.3 | 99.3 | 0.1 | <0.01 | 0.1 |
| March 2021 | 4.40 | 93.8 | 99.3 | 0.1 | <0.01 | 0.1 |
| April 2021 | 4.93 | 92.2 | 99.4 | 0.1 | <0.01 | 0.1 |
| May 2021 | 4.92 | 93.9 | 99.3 | 0.1 | <0.01 | 0.1 |
| June 2021 | 4.90 | 92.2 | 99.3 | 0.1 | <0.01 | 0.1 |
| July 2021 | 4.91 | 92.4 | 99.2 | 0.1 | <0.01 | 0.1 |
| August 2021 | 4.88 | 92.0 | 99.3 | 0.1 | <0.01 | 0.1 |
| September 2021 | 4.93 | 92.2 | 99.3 | 0.1 | <0.01 | 0.1 |
| October 2021 | 4.91 | 93.8 | 99.3 | 0.1 | <0.01 | 0.1 |
| November 2021 | 4.96 | 92.2 | 99.3 | 0.1 | <0.01 | 0.1 |
| December 2021 | 4.89 | 92.3 | 99.3 | 0.1 | <0.01 | 0.1 |

The new natural brown granulated or powdered slow sugar is nothing other than an equimolar mixture of glucose and fructose obtained by enzymatic or chemical hydrolysis of sucrose.

Hydrolysis of sucrose by an acid oxidant, which normally occurs partially, produced total sucrose.

The new natural brown granulated or powdered slow sugar of the present invention is characterized by a refractive index of 1.3408 and a polarization index according to the Z° unit of 60.0.

Blood sugar is measured according to the protocol of Oral Hyperglycemia (OGTT), by making a comparison based on analyses of the new natural brown slow sugar repeated on two subjects, and such analyses made it possible to note the hypoglycemic character compared to the natural white commercial sugar taken as a control and whose blood sugar is very high (FIG. 7).

The molecules of the esterified sugars of the new natural white slow sugar, that is to say esterified Sucrose, esterified Glucose and esterified Fructose in double molecule, homo or hetero are really and inevitably chemically synthesizable, namely:

### Glucose-Glucose, Fructose-Fructose, Glucose-Fructose and Sugar-Sugar.

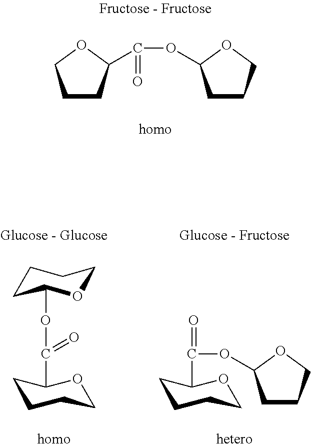

This is argued and proven by the results obtained from the Esterification Indexes IE, calculated by Saponification Index IS and Acid Index IA (IE=IS−IA) according to the French Pharmacopoeia tenth edition.

| ACID INDEX | | SAPONIFICATION INDEX | | ESTER INDEX |
|---|---|---|---|---|
| n(mL) | 1.35 | n1(mL) | 1.2 | IE = IS − IA |
| m(g) | 10 | n2(mL) | 1.31 | IS = 3.0855 |

| | | |
|---|---|---|
| m(g) | 1 | IA = 0.75735 |

| | | |
|---|---|---|
| IA = 0.75735 | IS = 3.0855 | IS = 2.32815 |

All the esterified sugar molecules designated in this invention bear the name SATTAR and are obtained by:

OXIDATION: All primary OH groups of sucrose, glucose and fructose are attacked more easily and are usually oxidized completely to the carboxylic acid state.

ESTERIFICATION: to subsequently ensure esterification, these molecules react with the sugar alcohol molecules, in homo-molecular or/and corresponding hetero-molecular (FIG. 5-2a) and (FIG. 5-2b).

1. A new natural brown slow sugar, granulated or powdered, characterized in that it is hypoglycemic, and process for its manufacture obtained through an industrial manufacturing process based on esterification using commercial white natural sugar and including a set of dried plant products, in accordance with the new molecule of the new slow natural brown sugar

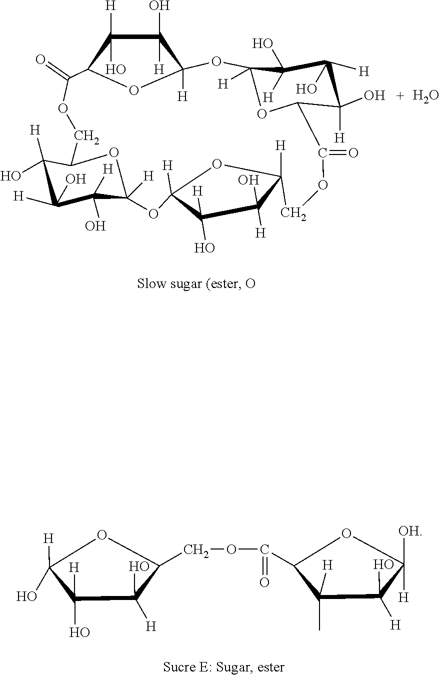

2. The new natural brown slow sugar of claim 1 retaining the sweetening power of commercial white natural sugar having a hypoglycemic character and whose glycemia is less than 1 gram, obtained by oxidation and esterification.

3. The new natural brown granulated or powdered slow sugar of claim 1 characterized by a refractive index of 1.3408 and a polarization unit Z° 60.0.

4. The new natural brown slow sugar of claim 1 which can be consumed by healthy and diabetic subjects.

5. The new natural brown slow sugar of claim 1 of anti-obesity by the effect of non-storage and the elimination of the surplus of sugar consumed thanks to the chemical balance esterification/hydrolysis acting in accordance with the slow secretion of insulin in the body.

6. The new natural brown slow sugar of claim 1 in that it is dietetic and without caloric intake, preventing dental caries by blocking the alcohol function (—OH) following the esterification of the sugar during its manufacture thanks to the acid oxidant and it can be consumed by diabetic subjects to slow down or/and prevent retinopathy and nephropathy and other pathological complications.

7. The new natural brown slow sugar of claim 1 in liquid solution, syrup, paste, cream, honey or caramel.

8. A method of manufacturing of new granulated or powdered natural brown slow sugar of claim 1 from commercial white natural sugar, in accordance with the following:
Mix two liters of demineralized water with an acid oxidant and a mixture of dried plants and seeds,
Heat and stir for 10 minutes in a bowl over a stove to reach 90 to 100 degrees Celsius,
Add 10 kg of commercial white natural sugar to the mixture,
Cook while stirring at a speed of 25 to 35 rpm,
Dry the molasses obtained in fresh air then in hot air for 30 to 45 minutes, stirring at a speed of 30 to 50 rpm.

9. The new natural brown slow sugar of claim 1 recommended for athletes practicing intense sports in that it carries characteristics offering endurance and preservation of muscles against cramps.

10. This is a property of the new natural brown granulated or powdered slow sugar of claim 1 used as raw material for the manufacture of products in liquid, pasty, creamy, honeyed or caramelized forms or other forms, for the preparation or production of pharmaceutical and parapharmaceutical products as well as for body care, well-being and cosmetic products.

11. The new natural brown slow sugar of claim 1 obtained by the chemical balance esterification/hydrolysis having the slow, limited and athermal characteristics.

12. The new natural brown slow sugar of claim 1 obtained by a reaction between sugar alcohol (sucrose) and an acidic sugar (saccharic acid) which is obtained by oxidation of the sugar alcohol.

13. A new natural brown slow sugar, slow when ingested into the intestine of the body characterized by its prolonged release LP in accordance with the secretion of insulin.

14. The new natural brown slow sugar in the form of esters, with regard to glucose, in accordance with the following molecule:

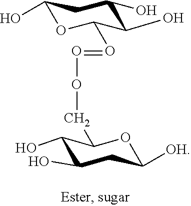

15. A new natural brown slow sugar chemically in the form of esters, with regard to fructose, in accordance with the following molecule:

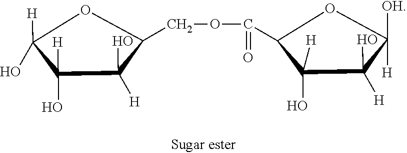

16. The esterification of two sugars can be homo i.e. esterification of each molecule separately or hetero i.e. esterification of the glucose molecule and the fructose molecule together

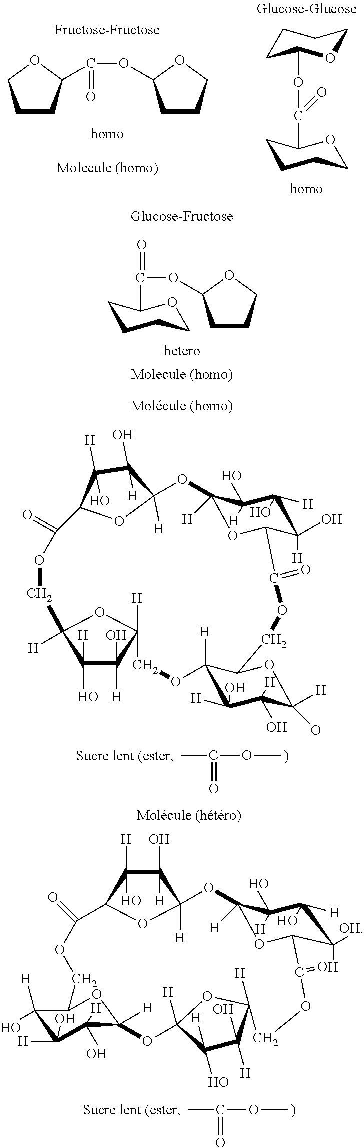

18. The oxidation of glucose into gluconic acid then the esterification between glucose and gluconic acid into an ester of two glucose molecules: 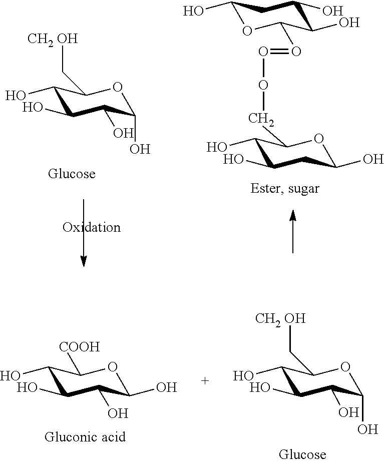
and
in the same way, the ester sugar for fructose is obtained

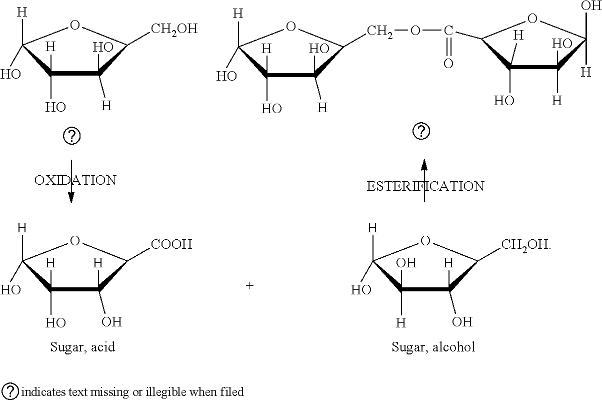

18. The new natural brown slow sugar of claim 2 which can be consumed by healthy and diabetic subjects.

19. The new natural brown slow sugar of claim 2 of anti-obesity by the effect of non-storage and the elimination of the surplus of sugar consumed thanks to the chemical balance esterification/hydrolysis acting in accordance with the slow secretion of insulin in the body.

20. The new natural brown slow sugar of claim 2 in that it is dietetic and without caloric intake, preventing dental caries by blocking the alcohol function (—OH) following the esterification of the sugar during its manufacture thanks to the acid oxidant and can be consumed by diabetic subjects to slow down or/and prevent retinopathy and nephropathy and other pathological complications.
